(19) United States
(12) Patent Application Publication
(10) Pub. No.: US 2009/0043255 A1
(43) Pub. Date: Feb. 12, 2009

(54) Manual Syringe

(75) Inventor: Ahmmed Ziah TAUFIG, Koln (DE)

Correspondence Address: GRIFFIN & SZIPL, PC, SUITE PH-1, 2300 NINTH STREET, SOUTH, ARLINGTON, VA, 22204, US

(73) Assignee: Ahmmed Ziah TAUFIG, Koln (DE)

(21) Appl. No.: 12/189,515

(22) Filed: Aug. 11, 2008

(30) Foreign Application Priority Data
Aug. 9, 2007 (DE) 20 2007 011 129.0

(51) Int. Cl. A61M 5/50

(52) U.S. Cl. 604/111

(57) ABSTRACT

A manual syringe (10) includes a disposable syringe cylinder (12), a disposable piston (14) and a disposable piston rod (16). The piston rod (16) includes a position encoder (18). A piston-position indicator means (22) coupled to the position encoder (18) is fixedly assigned to the syringe cylinder (12). The piston-position indicator means (22) is reusable and is adapted to be detached from the syringe cylinder (12).

This application claims priority from German Patent Application No. 20 2007 011 129.0, filed Aug. 9, 2007, the entire disclosure of which is incorporated herein by reference.

### FIELD OF THE INVENTION

The invention relates to a manual syringe comprising a disposable syringe cylinder, a piston and a piston rod.

### BACKGROUND OF THE INVENTION

Syringes of the above type are used particularly for the finely dosed application of a liquid substance. In the disposable syringes made of plastic which are used particularly in the medical field, the transparent syringe cylinder is provided with a linear scale on which the substance volume can be read as indicated by the piston position. The accuracy of the dosage is dependent, inter alia, on the accuracy of this reading. The optical resolution of such a linear cylinder scale is limited.

It is an object of the invention to provide a manual syringe having a piston-position indicator means with higher resolution.

### SUMMARY OF THE INVENTION

The above object is achieved by a manual syringe comprising (a) a disposable syringe cylinder (12); (b) a disposable piston (14); and (c) a disposable piston rod (16), wherein the piston rod (16) comprises a position encoder (18), a piston-position indicator means (22) coupled to the position encoder (18) that is fixedly assigned to the syringe cylinder (12), and wherein the piston-position indicator means (22) is reusable and is adapted to be detached from the syringe cylinder (12).

Thus, the manual syringe of the invention comprises a disposable piston rod provided with a position encoder. A piston-position indicator means coupled to the piston-rod position encoder is fixedly assigned to the disposable syringe cylinder. The piston-position indicator means is reusable and is configured to allow it to be detached from the disposable syringe cylinder.

The piston-position indicator means can be a high-resolution precision instrument that is manufactured with considerable expenditure and, therefore, should not be provided as a disposable article. While the syringe per se remains a disposable article, the piston-position indicator means is detachable from the syringe cylinder and thus is designed for virtually unlimited reuse. The position encoder and the piston-position indicator means can be realized in various manners, such as e.g. for mechanical or contactless and respectively electronic operation, and as analog and/or digital means.

By the provision of the piston-position indicator means, which is coupled or connected to the piston rod by the position encoder, it is made possible to realize an indicator means of virtually any desired resolution. This is of high importance particularly in cases where highly concentrated toxins are to be administered to a living body. By the high-resolution piston-position indicator means, the reading accuracy and the reliability of the reading are improved. The piston-position indicator means also offers the option to be set back to zero at any time, making it possible to avoid subtractions and additions which could cause errors to the user. If required, the resolution of the piston-position indicator means can be varied so that also the precision of the reading can be varied thereby. Depending on the resolution of the piston-position indicator means, the reading accuracy can be improved multiple times.

By the position encoder that is undetachably and—if possible—without play, arranged on the piston rod, the movement of the piston rod is transmitted to the piston-position indicator means without or nearly without play.

Preferably, use is made of a piston-position indicator means of the visual type, which optionally can have an acoustic indicator added thereto.

According to a preferred embodiment, the piston-position indicator means is provided as a dial gauge comprising a rotary shaft and a rotary hand. The rotary shaft is mechanically coupled to the piston rod and is driven by the latter. Preferably, the position encoder of the piston rod is a toothed rack, while the rotary shaft has a toothed wheel assigned thereto which is arranged for movement on the toothed rack. In this manner, a coupling of the piston rod to the piston-position indicator means is realized by a simple arrangement, which coupling can be produced in a simple manner and made to function without play.

The piston-position indicator means and, respectively, a dial gauge for use as an indicator means, can have assigned thereto a scroll wheel that is coupled to the dial-gauge rotary shaft and by which the advancement of the piston and of the piston rod can be performed in a highly sensitive manner and with fine resolution. It is left to the user's discretion whether the advancement of the piston is to be brought about directly at the piston rod or at the scroll wheel. Thus, it is rendered possible to manually dose the substance in an exact manner and to read the dosage precisely.

Preferably, the piston-position indicator means and the syringe cylinder are provided with a locking arrangement for coupling the syringe cylinder inclusive of the piston and the piston rod to the position encoder in a locking and detachable manner. The locking arrangement is preferably formed by an undercut syringe recess on the piston-position indicator means. The syringe recess has an e.g. cylindrical cross section and, when seen in cross section, comprises an enclosure extending along more than 180°.

The locking arrangement further comprises a corresponding axial fixing portion for a collar provided on the side of the syringe cylinder, resulting in the syringe cylinder being fixed to the piston-position indicator means also axially.

An embodiment of the invention will be explained in greater detail hereinafter with reference to the drawings.

### BRIEF DESCRIPTION OF THE DRAWINGS

In the drawings

FIG. 1 is a lateral view of a manual syringe together with a piston-position indicator means from the mounting side;

FIG. 2 is a view of the syringe from the indication side;

FIG. 3 is a cross-sectional view III-III of the piston-position indicator means of the syringe illustrated in FIGS. 1 and 2;

FIG. 4 is a cross-sectional view IV-IV of the piston-position indicator means of the syringe illustrated in FIGS. 1 and 2;

FIG. 5 is a cross-sectional view V-V of the piston-position indicator means of the syringe illustrated in FIGS. 1 and 2; and

FIG. 6 is a lateral view of the removed piston-position indicator means as seen from the mounting side.

### DETAILED DESCRIPTION OF A PREFERRED EMBODIMENT

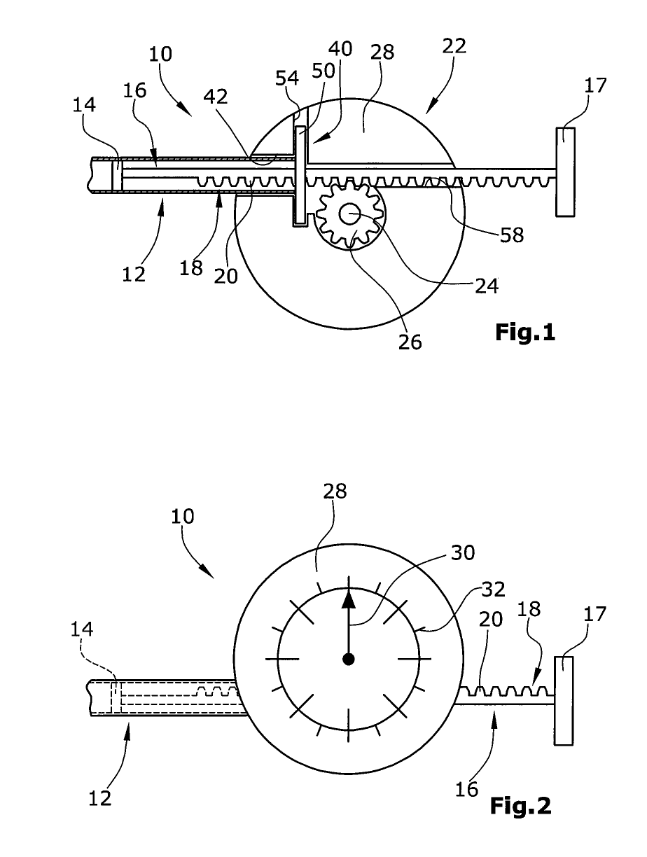

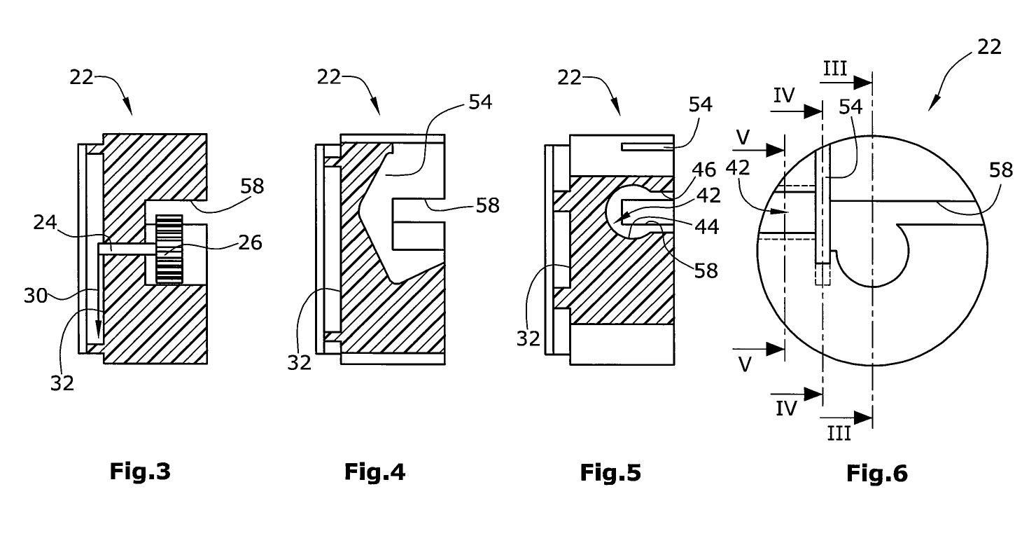

Illustrated in FIGS. 1 and 2 is a manual syringe 10 as used e.g. in the medical field for application of a substrate, e.g. a medicant. Syringe 10 comprises a disposable syringe cylinder 12 accommodating a piston 14 arranged on a piston rod 16. Piston rod 16 comprises a toothed rack 20 as a position encoder 18. Syringe cylinder 12 and piston 14 inclusive of toothed rack 20 are disposable articles made of plastic, which after one usage will not be reused but will be disposed of.

Syringe 10 further comprises a piston-position indicator means 22, which in FIGS. 1 and 2 is shown in the condition as mounted to syringe cylinder 12. Piston-position indicator means 22 is designed as a visual indicator means in the form of a dial gauge and comprises, inter alia, a substantially cylindrical base body 28, a rotary shaft 24, a toothed wheel 26 seated on rotary shaft 24, as well as a pointer 30 arranged on a circular scale 32. The toothing of toothed wheel 26 and of toothed rack 20 as well as the length of toothed rack 20 are selected in such a manner that the pointer 30 of the dial gauge will perform four rotations along the overall piston path. The number of rotations should in each case be in a range from one full rotation to about six rotations.

The piston-position indicator means 22 and the syringe cylinder 12 are provided with a locking arrangement 40 by which the disposable syringe cylinder 12 is axially and radially as well as rotatably fixed to and respectively in the piston-position indicator means 22 in a locked and detachable manner. The locking arrangement 40 is substantially formed by an undercut syringe recess 42 of a hollow cylindrical shape. Syringe recess 42 substantially consists of a hollow cylinder 44, which is symmetrically slitted in the longitudinal direction and has an angle of contact from 180° to 260°. Since the base body 28 of piston-position indicator means 22 is made from plastic of a certain elasticity, which also holds true for syringe cylinder 12, syringe cylinder 12 can be radially inserted or removed through the longitudinal groove 46 of syringe recess 42 while effecting a locking and respectively release action.

A cylinder collar 50 of syringe cylinder 12 is seated in a corresponding transverse groove 54 of base body 28 of piston-position indicator means 22. Thereby, syringe cylinder 12 is axially fixed to piston-position indicator means 22.

Beyond syringe recess 42 relative to transverse groove 54, a piston-rod groove 58 for receiving the piston rod 16 is formed in linear extension of the syringe recess 42.

The syringe 10 is to be used as follows: First, the disposable syringe cylinder 12, together with its inserted piston 14 and the piston rod 16 integrally connected to the piston, will be retracted, i.e. the substance will be sucked into the syringe cylinder 12 by pulling the piston rod 16. Thereafter, syringe cylinder 12 will be radially moved into syringe recess 42 until syringe cylinder 12 becomes locked in the mounting position in the undercut syringe recess 42. In this position, also the toothed wheel 26 will engage the toothed rack 20 of piston rod 16. The piston-position indicator means 22 can be provided with a reset mechanism or, for instance, with a correspondingly rotatable dial gauge 32 for setting the pointer to zero.

Now, by seizing the piston-rod end plate 17 and the syringe collar 50, the user can press the syringe 10 together, i.e. shift the piston 14 into the syringe cylinder 12, as he or she is accustomed to do. The circular dial 32 will allow the user to perform a highly accurate reading of the quantity of the substance administered in the process.

Subsequent to complete application of the substance, the piston-position indicator means 22 will be radially removed from the syringe cylinder 12 again, the syringe cylinder 12 together with piston 14 and piston rod 16 will be disposed of, and the piston-position indicator means 22 can be subjected to a sterilization process, if required. After sterilization, the piston-position indicator means 22 can be employed for a further administering process using a new syringe cylinder 12.

Although the invention has been described and illustrated with reference to specific illustrative embodiments thereof, it is not intended that the invention be limited to those illustrative embodiments. Those skilled in the art will recognize that variations and modifications can be made without departing from the true scope of the invention as defined by the claims that follow. It is therefore intended to include within the invention all such variations and modifications as fall within the scope of the appended claims and equivalents thereof.

1. A manual syringe, comprising:
(a) a disposable syringe cylinder;
(b) a disposable piston, and
(c) a disposable piston rod connected to the disposable piston, wherein
the piston rod comprises
i. a position encoder; and
ii. a piston-position indicator means coupled to the position encoder and that is fixedly assigned to the syringe cylinder, and
wherein
the piston-position indicator means is reusable and is detachable from the syringe cylinder.

2. The manual syringe according to claim 1, wherein the piston-position indicator means is an indicator means of the visual type, or the acoustic type, or the visual and acoustic type.

3. The manual syringe according to claim 2, wherein the piston-position indicator means is a dial gauge comprising a rotary shaft and a pointer.

4. The manual syringe according to claim 1, wherein the position encoder is a toothed rack engaging a toothed wheel on a rotary shaft of the piston-position indicator means.

5. The manual syringe according to claim 1, wherein the piston-position indicator means and the syringe cylinder are provided with a locking arrangement.

6. The manual syringe according to claim 5, wherein the locking arrangement is formed by an undercut syringe recess on the piston-position indicator means.

7. A manual disposable syringe without piston-position indicator means, the manual disposable syringe comprising:
(a) a disposable syringe cylinder;
(b) a disposable piston disposed inside of the disposable syringe cylinder; and
(c) a disposable piston rod connected to the disposable piston wherein the piston rod comprises
i. a position encoder.

8. A piston-position indicator means for a disposable syringe, wherein the syringe comprises a disposable piston and a disposable piston rod connected to the piston, and the piston rod comprises a position encoder, and said piston-position indicator means is coupled to the position encoder and is fixedly assigned to a syringe cylinder of the syringe; wherein the piston-position indicator means is reusable and is detachable from the syringe cylinder.
